(19) United States
(12) Patent Application Publication
(10) Pub. No.: US 2017/0360698 A1
(43) Pub. Date: Dec. 21, 2017

(54) Multi-Component Bolus

(71) Applicant: Animax Limited, Suffolk (GB)

(72) Inventors: William Leslie Porter, Norfolk (GB); Elizabeth Berry, Suffolk (GB)

(21) Appl. No.: 15/622,506

(22) Filed: Jun. 14, 2017

(30) Foreign Application Priority Data
Jun. 15, 2016 (GB) 1610409.3

(51) Int. Cl. A61K 9/00; A61K 47/02; A61K 33/18; A23K 20/20; A61K 33/04; A23K 50/10; A61K 47/30; A61K 33/24

(52) U.S. Cl. CPC A61K 9/0068; A61K 47/30; A61K 47/02; A61K 33/24; A23K 20/30; A61K 33/04; A23K 50/10; A61K 33/18

(57) ABSTRACT

Disclosed is a bolus for oral administration to an animal, the bolus comprising at least one beneficial substance to be delivered to the animal, the bolus comprising two components which are adhered together by a water-soluble adhesive or sealant, such that the two components are separable in vivo, and wherein at least one of the aforesaid components is frangible in vivo, the frangible component being initially stabilised by the adherence thereto of the other of the aforesaid components, such that separation of the two components in vivo facilitates the breakage of the frangible component.

### FIELD OF THE INVENTION

This invention relates to a novel multi-component bolus, and a method of making the same.

### BACKGROUND OF THE INVENTION

It is conventional, in animal husbandry, to provide beneficial substances in a solid bolus form, to be administered orally. The beneficial substance may be, for example, a therapeutic or prophylactic drug (e.g. an anti-parasitic or anti-microbial agent), or a dietary supplement (e.g. one or more trace minerals; or compounds to promote weight gain or regulate one or more particular aspects of metabolism).

The solid boluses are typically administered by means of a dosing gun, which forces the bolus towards the rear of the animal's throat, whence the bolus is swallowed into the digestive tract. Boluses are frequently used to dose cattle and sheep, and are sized accordingly, typically with a generally cylindrical shape to facilitate their passage down the oesophagus and into the digestive system. The beneficial substance is typically incorporated into the bolus and is dissolved or leaches out therefrom in vivo. This leaching may be accelerated by abrasion of the exterior surface of the bolus on prolonged and/or repeated contact with the wall of the animal's digestive tract.

Many different types of bolus are known. For example GB 2376630 discloses a segmented bolus, in which a single bolus is formed from a plurality of disk-like segments, assembled to form a generally cylindrical bolus. The segments may be identical, or may comprise different beneficial substances.

GB 2510826 discloses an arrangement for the simultaneous administration of multiple bolus forms in a single dosing action. The document discloses, inter alia, two generally hemispherical or arch-shaped boluses (which fit together within a dosing gun), defining a narrow circular cylindrical volume there between, which volume can accommodate a third, cylindrical, bolus form.

In addition to the beneficial substance, it is conventional for orally-administered boluses to comprise one or more of the following: a matrix or binder material; a relatively dense ballast material; a protective coating.

### SUMMARY OF THE INVENTION

In a first aspect the invention provides a bolus for oral administration to an animal, the bolus comprising at least one beneficial substance to be delivered to the animal, the bolus comprising two components which are adhered together by a water-soluble adhesive or sealant, such that the two components are separable in vivo, and wherein at least one of the aforesaid components is frangible in vivo, the frangible component being initially stabilised by the adherence thereto of the other of the aforesaid components, such that separation of the two components in vivo facilitates the breakage of the frangible component.

Conveniently the frangible component comprises one or more frangible zones or regions. The frangible zone or region may be structurally weaker than the bulk of the frangible component due to, for example, a different composition and/or being relatively thin. Preferably the frangible zone or region is relatively thin, i.e. is thin relative to most of the rest of frangible component. The frangible zone or region may run along substantially the entire length of the frangible component.

In a preferred embodiment the two components co-operate to form a generally cylindrical bolus, which may be swallowed by an animal such as a sheep, goat or cow.

In a preferred embodiment, one of the two components comprises an indented portion, such as a deep groove or recess, into which the other component of the bolus wholly or partially inserts or penetrates. Advantageously, the other component is substantially wholly located within the groove or recess in the frangible component. More especially, the frangible component may comprise a frangible region or zone which extends substantially along a long axis of the component, the frangible region or zone being relatively thin, the relative thinness of the frangible region or zone typically resulting from the presence of a deep longitudinal groove or recess formed in the frangible component. In such an embodiment, the other component is substantially located along and within the deep longitudinal groove, the presence of the other component serving to stabilise and protect the frangible region or zone.

In a preferred embodiment, the frangible component has an almost complete circular cross section but with a ‘wedge’-shape almost bisecting the circle to produce a ‘U’-type shape, with the two arms being relatively thick, and being joined by a cross-piece which is relatively thin (i.e. thin relative to the two arms) and which represents a frangible region or zone of the frangible component. The frangible component is stabilised by the presence of the other component of the bolus between the arms of the U-shape, and which is adhered to the frangible component by the water-soluble adhesive. It will be appreciated that, in practice, other parts of the bolus might be frangible in vivo. The term “frangible” as used herein, is intended to indicate that the greatest frangibility is associated with the ‘frangible’ component, and especially the frangible zone or region thereof.

The beneficial substance present in the bolus may be, for example, a therapeutic or prophylactic drug, or may be a dietary supplement, such as a trace mineral or the like, or any combination thereof. Trace minerals which may be present in the bolus include, inter alia, any one or more of the following: copper, zinc, manganese, selenium, iodine and cobalt. These substances may be present in elemental form (especially for copper and zinc) or may be present as compounds (such as a cobalt salt e.g. cobalt sulfate monohydrate; potassium iodide, calcium iodate; and sodium selenite, or sodium selenate).

The two components of the bolus of the invention may have essentially identical composition or may be different. In particular, the two components may comprise different beneficial substances or different mixtures of beneficial substances. For example, the applicant has found that it is beneficial to separate the salts of trace elements into distinctly different regions, as it has been determined that releasing/leaching may not be optimal if placed into the same bolus. Accordingly, in one preferred embodiment, one component of the bolus comprises a cobalt salt, and the other component comprises a selenium compound such as sodium selenite.

As well as preferably having different compositions, in a preferred embodiment, one of the components of the bolus is more massive than the other. As mentioned above, in a particular embodiment, the bolus may comprise a generally U-shaped component (which is frangible) and a second component inserted between the arms of the “U”. In this embodiment the U component is relatively massive, having approximately 1.5-2.5 times the mass of the smaller component inserted between the arms.

Again in a preferred embodiment, at least one of the components of the bolus comprises one or more grooves or recesses on its exterior surface. Preferably these are provided on the frangible component. Preferably a plurality of such grooves or recesses are provided. Advantageously two or more longitudinal grooves are formed on the exterior surface of the bolus component parallel to the long axis thereof. In a particularly preferred embodiment there is at least one groove either side of the frangible region or zone such that, when the frangible component breaks, each of the constituent pieces of the frangible component comprises at least one longitudinal groove. More especially, the plurality of exterior grooves may comprise at least two different depths and/or widths. For example, where the grooves have a section forming a segment of circle, it is desirable that a first groove may be a section with a particular radius of curvature and a second groove may be of a section with a different radius of curvature.

The two components of the bolus are sealed together by a water-soluble adhesive or sealant. This adhesive or sealant is dissolved in the digestive tract of the animal once the bolus is administered. The water-soluble adhesive or sealant may be any suitable non-toxic material. Examples include wetted rice paper, sugar-glass preparations and black treacle/molasses-based adhesives. Conveniently the adhesive or sealant should be able to withstand exposure to a temperature of about 80° C. for a short period of time (1 minute or less) without substantial loss of integrity, as these conditions may be employed during production of the bolus.

Advantageously the bolus is coated with a layer of water-impermeable material. The function of this is, at least in part, to protect the water-soluble adhesive from exposure to water or moisture in the environment prior to administration to the animal. The water-impermeable layer is preferably soft e.g. a wax (such as carnauba wax, paraffin wax or beeswax), or poly ethyl vinyl acetate and preferably quite thin (e.g. 1-2 mm), such that the layer is readily removed from the bolus in vivo by abrasion, thus exposing the water-soluble adhesive to the action of the aqueous environment in the animal's digestive tract.

It will be appreciated that the thickness of the water-impermeable coating, and its toughness, can be selected so as to vary the length of time taken for the coating to be removed from the bolus, which will impact on the time before the water-soluble adhesive is exposed to the aqueous environment.

In addition, the amount of adhesive, and/or the location(s) to which the adhesive is applied, can also be selected to vary the degree of exposure of the adhesive to the aqueous environment and to vary the amount of time taken for sufficient of the adhesive to be dissolved to allow separation of the stabilising bolus component from the frangible bolus component. This in turn controls the amount of time, after administration to the animal, required for the frangible component to break, and so can vary the rate of release, and the release profile (over time) of the beneficial substance from the bolus. In particular, it may be possible to retard or “even out” the initial surge of release of beneficial substance which is frequently observed with conventional boluses.

By having two or more frangible components, with differing degrees of frangibility, it may be possible to provide further regulation over the rate of release of the beneficial substance.

The bolus of the invention typically comprises a matrix and/or binder material, the beneficial substance or substances being typically incorporated within the binder and/or matrix, typically in a substantially homogeneous mixture. Materials suitable for use as a binder or matrix in a bolus are well-known to those skilled in the art and typically have no, or very little, solubility in water (and is thus substantially insoluble in the animal's digestive tract). Suitable materials include, inter alia, resin or rosin (especially a pine rosin), poly ethyl vinyl acetate, or a wax, or any mixture of the foregoing may be used, as may also be a finely porous porcelain or other ceramic material.

The present invention is especially, but not only, applicable to boluses which release the beneficial substance or substances over a prolonged period (e.g. typically over at least two months or more, preferably over at least four months, more preferably over at least six months).

In a typical embodiment, the bolus of the invention is adapted and configured for oral administration to a ruminant animal, especially a cow. The bolus is thus advantageously configured and adapted to be retained in the rumen or the reticulum (sometimes referred to collectively as the reticulorumen) of the ruminant animal, and the inclusion of a ballast component to increase the density of the bolus is desirable, to reduce the likelihood of the bolus being regurgitated (especially after it has been in the animal for a month or more, and so is reduced in size). The ballast material may comprise, for example, metallic zinc and/or copper, which may also act as a valuable source of the element for the animal as it gradually dissolves from the bolus.

Although the preceding description primarily concerns embodiments of the invention comprising two components, and such embodiments are generally preferred, it is perfectly feasible for the bolus of the invention to comprise three, four or more components. Where there are three or more components of the bolus, there may be one frangible component and two relatively non-frangible components, or there may be two frangible components and one relatively non-frangible component. Additionally, or alternatively, one of the components of the bolus may comprise more than one frangible region or zone such that, in vivo, the frangible component may fragment into more than two pieces.

The beneficial substance or substances may be incorporated into a specific part or parts of the bolus, or into a specific part or parts of one or more of the bolus components, or may be substantially homogenously incorporated throughout the matrix or binder of the bolus or a single bolus component, or multiple components.

The beneficial substance or substances are typically released from the bolus by leaching or dissolution. The matrix or binder which typically makes up the majority of the volume of the bolus is generally a substance which can be eroded or degraded by abrasion with the surfaces of the animal's digestive tract, which exposes fresh portions of the bolus for leaching or dissolution of the beneficial substance(s).

In a second aspect, the invention provides a method of making a bolus in accordance with the first aspect defined above, the method comprising the steps of: forming a first bolus component; forming a second bolus component; and adhering the first and second components together by means of a water-soluble adhesive; wherein at least one of said bolus components is frangible in vivo, and is stabilised by the adherence of the other component.

The methods of manufacturing the bolus components may be substantially or wholly conventional. Typically a mixture will be formed, comprising one or more bolus matrix or binder materials and the beneficial substance. This mixture is then hardened if necessary, and formed into a bolus component by moulding, extrusion and compression or similar such process. The two or more separate bolus components are then joined to form a single bolus, the bolus components being held together by application of a water-soluble adhesive. The adhesive may require a curing process or exposure to particular conditions (e.g. heating briefly) to undergo curing.

Once the bolus components have been assembled, the entire bolus is preferably coated with a thin water-impermeable layer such as a wax or the like. Methods by which the coating may be applied to the bolus include, but are not limited to, dipping in molten or dissolved coating material, spraying, powder coating, and application by means of a coating pan.

The various features of the invention will now be further described by way of illustrative embodiment and with reference to the accompanying drawings, in which:

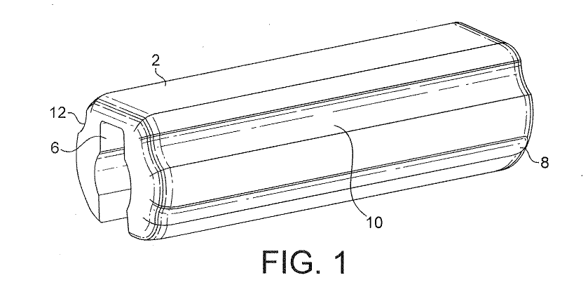

FIG. 1 shows a perspective view of one embodiment of a first component of a multi-component bolus in accordance with the invention;

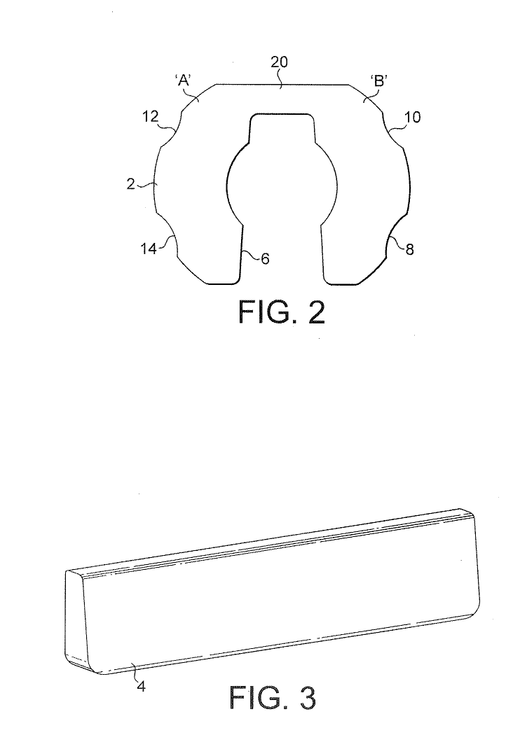

FIG. 2 shows a sectional view of the component shown in FIG. 1;

FIG. 3 is a perspective view of an embodiment of a second component, which co-operates with the component illustrated in FIGS. 1 and 2, to form a multi-component bolus in accordance of the invention.

For the avoidance of doubt, it is hereby expressly stated that the features described herein as “preferred”, “preferable”, “convenient”, “advantageous” and the like may be present in embodiments of the invention in isolation, or in combination with any other features so described, unless the context dictates otherwise.

### DETAILED DESCRIPTION OF AN EMBODIMENT

Referring to FIGS. 1 and 3, there is provided a two component solid bolus for delivering a beneficial substance to a farm animal such as a sheep or cow. The components 2 and 4 co-operate to form a substantially circular-section cylinder. Component 2 has a U section and may be referred to for present purposes as the “sheath component”. Component 4 is a blade-like wedge portion and may be referred to for present purposes as the “wedge component”.

Sheath component 2 is formed with a deep longitudinal recess or groove 6 running along the long axis of the component. The wedge component 4 is substantially entirely accommodated within the recess or groove 6, and is shaped and dimensioned accordingly. The two components 2, 4 are joined by a water-soluble adhesive or sealand, which is applied over all, most or part (as desired) of the co-operating surfaces of the two components. The outside of the bolus (once the two components are joined together) is coated with a thin layer of water-impermeable wax.

The sheath component 2 comprises sodium selenite, potassium iodide and calcium iodate homogeneously distributed within a rosin matrix/binder. The concentration of the beneficial substances in this embodiment are sufficient to provide 1 gram of selenium and 7.8 grams of iodine in bioavailable form.

The wedge component 4 comprises cobalt sulfate monohydrate, at a concentration sufficient to provide 1 gram of cobalt per bolus. In addition, each component of the bolus comprises a zinc and/or copper metal ballast agent, which serves to help keep the bolus components within the animal's digestive tract rather than being regurgitated. The total mass of the cobalt-containing bolus may typically be in the region of ˜40 gms.

As to the dimensions of the bolus, the sheath component 2 is 84 mm long, 24 mm high and, at its widest, 30-31 mm wide. The recess or groove 6 runs the entire length of the sheath component and is about 20 mm deep, such that it is almost as deep as the sheath component is high. The recess or groove 6 is about 10-11 mm wide.

As well as the recess or groove 6 on the interior of the component 2, the component has four parallel grooves on the exterior surface, three of which, 8, 10, 12 are visible in FIG. 1. The fourth groove 14, is visible in FIG. 2. The grooves in the exterior surface are longitudinal grooves, parallel to the long axis of the component, and parallel to the interior recess or groove 6.

With reference to FIG. 2 it can be seen that, in section, component 2 has the appearance of a U. The two pointing arms ‘A’ and ‘B’ are relatively thick and are joined by a relatively thin bridging portion, which constitutes a frangible region or zone 20. The frangible zone 20 runs along the length of the bolus component such that, in vivo, when the stabilising wedge component 4 falls out from the between the arms A and B, the frangible sheath component 2 tends to rupture along its length, releasing the two arms A and B.

The longitudinal grooves 8-14 in the exterior surface of the component are symmetrically positioned, such that when the component breaks along it length, each of the resulting pieces comprises two grooves (8 and 10 in arm B; and 12 and 14 in arm A). The grooves 10 and 12 have a section with a radius of curvature of 5.1 mm, whilst grooves 8 and 14 are a little wider having a section with a radius of curvature of 5.6 mm. The function of the exterior grooves 8-14 is to allow the wax-coating of different parts of the bolus to be eroded at different rates. Thus, the portions of the wax directly beneath the grooves 8-14 are protected to some extent from the abrasion which takes place in vivo, whilst the surrounding portions are relatively protruding and so will be eroded, and their incorporated beneficial substance released, more quickly. This feature is already described in our GB patent no. 2510826.

In the actual bolus, the wedge component 4 is inserted into, and substantially entirely accommodated within, the groove or recess 6 in the sheath component 2, such that the bolus as a whole has a reasonably smooth, flush surface at the junction of the two components. The two components are held together in the bolus, ex vivo, by a water soluble adhesive or sealant, such as rice paper, which may be applied to the abutting surface of one or other, or both, of the bolus components, and the bolus as a whole is coated with a thin water-impermeable coating, such as a wax, the function of which, at least in part, is to protect the water-soluble adhesive from exposure to moisture prior to administration to an animal.

Once in vivo, the water-impermeable coating is removed from the bolus by abrasion with the wall of the animal's digestive tract, exposing the water-soluble adhesive to the aqueous environment of the digestive tract.

1. A bolus for oral administration to an animal, the bolus comprising at least one beneficial substance to be delivered to the animal, the bolus comprising two components which are adhered together by a water-soluble adhesive or sealant, such that the two components are separable in vivo, and wherein at least one of the aforesaid components is frangible in vivo, the frangible component being initially stabilised by the adherence thereto of the other of the aforesaid components, such that separation of the two components in vivo facilitates the breakage of the frangible component.

2. The bolus in accordance with claim 1, wherein the bolus comprises two or more frangible components.

3. The bolus in accordance with claim 1, wherein the frangible component comprises one or more frangible zones or regions.

4. The bolus in accordance with claim 3, wherein the frangible zone or region is structurally weaker than the bulk of the frangible component.

5. The bolus in accordance with claim 4, wherein the frangible zone or region is relatively thin.

6. The bolus in accordance with claim 3, wherein a frangible zone or region runs along substantially the entire length of the frangible component.

7. The bolus in accordance with claim 1, wherein one of the bolus components is wholly or partially inserted into the other bolus component.

8. The bolus in accordance with claim 7, wherein one of the bolus components is wholly or partially inserted into a longitudinal groove or recess formed in the frangible component.

9. The bolus in accordance with claim 1, wherein the bolus components comprise different respective beneficial substance or substances.

10. The bolus in accordance with claim 9, wherein one bolus component comprises a cobalt-containing compound and the other bolus component comprises a selenium-containing compound.

11. A method of making a bolus in accordance with claim 1, the method comprising the steps of: forming a first bolus component; forming a second bolus component; and adhering the first and second components together by means of a water-soluble adhesive; wherein at least one of said bolus components is frangible in vivo, and is stabilised by the adherence of the other component.
